(19) United States
(12) Patent Application Publication
(10) Pub. No.: US 2009/0192593 A1
(43) Pub. Date: Jul. 30, 2009

(54) Stent for Delivery a Therapeutic Agent from a Side Surface of a Stent StSrut

(75) Inventors: Michael P. Meyer, Richfield, MN (US); James E. McGovern, Eden Prairie, MN (US); Daniel Gregorich, St. Louis Park, MN (US); Shawn Sorenson, Maple Grove, MN (US); Aaron Foss, Plymouth, MN (US)

Correspondence Address: VIDAS, ARRETT & STEINKRAUS, P.A., SUITE 400, 6640 SHADY OAK ROAD, EDEN PRAIRIE, MN, 55344, US

(73) Assignee: Boston Scientific Scimed, Inc., Maple Grove, MN (US)

(21) Appl. No.: 12/353,509

(22) Filed: Jan. 14, 2009

Related U.S. Application Data
(60) Provisional application No. 61/023,142, filed on Jan. 24, 2008.

(51) Int. Cl. A61F 2/82

(52) U.S. Cl. 623/1.42

(57) ABSTRACT

Described herein are implantable medical devices, such as implantable or intravascular stents, for delivering a therapeutic agent, and methods for making such medical devices. In one embodiment, the medical device comprises a stent having a plurality of struts, at least one of which has a cavity disposed therein. A therapeutic agent is delivered from the cavity through and opening in a strut surface. In another embodiment, the medical device is a stent having a coating disposed on the side surface(s) of at least one strut for deliver of a therapeutic agent from the coating.

### CROSS-REFERENCE TO RELATED APPLICATIONS

This application claims priority from Provisional Application No. 61/023,142, filed Jan. 24, 2008, the contents of which is hereby incorporated by reference

### FIELD OF INVENTION

Described herein are implantable medical devices, such as implantable or intravascular stents, for delivering a therapeutic agent, and methods for making such medical devices. In one embodiment, the medical device comprises a stent having a plurality of struts, at least one of which has a cavity disposed therein. A therapeutic agent is delivered from the cavity through an opening in a strut surface. In another embodiment, the medical device is a stent having a coating disposed on the side surface(s) of at least one strut for deliver of a therapeutic agent from the coating.

### BACKGROUND

Medical devices have been used to deliver therapeutic agents locally to the body tissue of a patient. For example, stents having a coating containing a therapeutic agent, such as an anti-restenosis agent, have been used in treating or preventing restenosis. Currently, such medical device coatings include a therapeutic agent alone of a combination of a therapeutic agent and a polymer. Some polymer coating compositions, however, do not actually adhere to the surface of the medical device. In order to ensure that the coating compositions remain on the surface, the area of the medical device that is coated, such as a stent strut, is encapsulated with the coating composition. However, since the polymer does not adhere to the medical device, the coating composition is susceptible to deformation and damage during loading, deployment and implantation of the medical device. Any damage to the polymer coating may alter the therapeutic agent release profile and can lead to an undesirable increase or decrease in the therapeutic agent release rate.

Furthermore, by encapsulating a stent strut with a coating comprising a therapeutic agent, an amount of the therapeutic agent greater than the desired amount may be delivered. For instance, if the therapeutic agent is an anti-restenosis agent, by applying a coating containing such an agent to all surfaces of the strut, including the luminal surface, may result in the unnecessary delivery of the anti-restenosis agent to the bloodstream.

Accordingly, there is a need for medical devices that can release an effective amount of a therapeutic agent in a desired manner while avoiding the disadvantages of current medical devices for delivering a therapeutic agent. Additionally, there is a need for methods of making such medical devices.

### SUMMARY

These and other objectives are addressed by the medical devices described herein. In one embodiment, the medical device comprises an implantable stent, which comprises a stent sidewall structure comprising a plurality of struts and gaps in the sidewall structure. At least one strut comprises an abluminal surface, a luminal surface, and a first side surface. The strut comprises a first material. Also, the at least one strut comprises at least one cavity disposed therein. The cavity has a first opening that is in fluid communication with the abluminal surface and a second opening that is in fluid communication with the first side surface. A therapeutic agent can be disposed in the cavity. Furthermore, a coating is disposed over at least a portion of the first opening of the cavity that is in fluid communication with the abluminal surface. The second opening of the cavity, which is in fluid communication with the side surface, is at least partially exposed.

In another embodiment, the medical device comprises an implantable stent that comprises a stent sidewall structure comprising a plurality of struts and gaps in the sidewall structure. At least one strut comprises an abluminal surface, a luminal surface, and a first side surface. Also, the at least one strut comprises at least one cavity disposed therein, in which the cavity has a first opening that is in fluid communication with the first side surface and the first opening of the cavity is at least partially exposed. The cavity does not have any opening that is in fluid communication with the abluminal surface or the luminal surface. Also a therapeutic agent is disposed in the cavity.

In addition, in one embodiment, the medical device comprises an implantable stent that comprises a stent sidewall structure comprising a plurality of struts and gaps in the sidewall structure. At least one of the struts comprises an abluminal surface, a luminal surface, and a first side surface. A first coating is disposed on at least a portion of the first side surface and the abluminal and luminal surfaces are substantially free of any coating.

Also described herein are methods for making medical devices. In one embodiment, the method is one for making an implantable stent. The method comprises the step of providing a tube having a tubular wall having a longitudinal axis, in which the tubular wall comprises an abluminal surface and luminal surface. There is at least one groove disposed in the abluminal surface of the tubular wall and the groove does not extend through the tubular wall to the luminal surface. A therapeutic agent is disposed in at least a portion of the groove. A coating is disposed over at least a portion of the abluminal surface such that at least a portion of the therapeutic agent disposed in the groove is covered by the coating. A stent is formed from the tube that has the coating disposed on the abluminal surface. The stent has a stent sidewall structure comprising a plurality of struts and gaps, wherein at least one of the struts comprises an abluminal surface, a luminal surface, and a first side surface. Also, the strut has a cavity that comprises a portion of the groove containing the therapeutic agent, and the cavity that comprises a portion of the groove containing the therapeutic agent, and the cavity has an opening, which is at least partially exposed, that is in fluid communication with the first side surface. Alternatively, instead of disposing a therapeutic agent in the groove, a filler material is disposed in the groove. After the stent is formed, the filler material is removed from the cavity and a therapeutic agent is disposed in the cavity.

In another embodiment, the method for making an implantable stent comprises providing a flat sheet of a material having a sheet wall. The sheet wall comprises a first surface and second surface. The method comprises disposing at least one groove in the first surface of the sheet wall, wherein the groove does not extend through the sheet wall to the second surface. A therapeutic agent is then disposed in at least a portion of the groove. A coating is disposed over at least a portion of the first surface such that at least a portion of the therapeutic agent disposed in the groove is covered by the coating. A sidewall structure is formed from the sheet, having the coating disposed on the first surface, by removing portions of the coated sheet. The a sidewall structure comprising a plurality of struts and gaps, wherein at least one of the struts comprises a top surface, a bottom surface, a first side surface and a cavity. The cavity comprises a portion of the groove containing the therapeutic agent, and an opening that is in fluid communication with the first side surface. The opening is at least partially exposed. Instead of disposing a therapeutic agent in the groove, a filler material can be disposed in the groove. After the sidewall structure is formed, the filler material is removed from the cavity and a therapeutic agent is disposed in the cavity.

In another method for making an implantable stent, the method comprises providing a stent having a stent sidewall structure comprising a plurality of struts and gaps in the sidewall structure. There is at least one strut comprises an abluminal surface, a luminal surface, and a first side surface. Also, at least one vacity is disposed in the strut and the cavity has a first opening that is in fluid communication with the first side surface and a second opening that is in fluid communication with the abluminal surface or the luminal surface. The method further comprises disposing a therapeutic agent in the cavity and applying a material over the second opening and any other opening so that the cavity is not in fluid communication with the abluminal surface or luminal surface. However, the first opening is at least partially exposed.

In a further embodiment, the method for making an implantable stent comprises providing a stent having a stent sidewall structure comprising a plurality of struts and gaps in the sidewall structure. At least one strut comprises an abluminal surface, a luminal surface, and a first side surface. There is at least one cavity disposed in the strut that has a first opening that is in fluid communication with the first side surface. The cavity is not in fluid communication with the abluminal surface or the luminal surface. The method further comprises disposing a therapeutic agent in the cavity and allowing the first opening to be at least partially exposed.

Moreover, in another embodiment, the method for making an implantable stent comprises providing a stent having a stent sidewall structure comprising a plurality of struts and gaps in the sidewall structure, wherein at least one strut comprises an abluminal surface, a luminal surface, and a first side surface. The stent is disposed on a mandrel. A coating composition comprising a therapeutic agent is applied onto a flat surface. The stent disposed on the mandrel is rolled in the coating composition applied to the flat surface to apply the coating composition to the first side surface of the strut. Steps are taken to ensure that the abluminal surface and the luminal surface are substantially free of the coating composition.

### BRIEF DESCRIPTION OF THE DRAWINGS

The embodiments described herein will be explained with reference to the following drawings.

FIG. 1 shows a perspective view of an example of a medical device having a sidewall structure comprising a plurality of struts and gaps or opening and at least one cavity disposed in at least one strut.

FIG. 2A shows a perspective view of a strut of one embodiment of a medical device, such as a stent.

FIG. 2B shows a portion of the strut of FIG. 2A located between lines A-A and B-B when the portion is placed in a vessel.

FIG. 2C shoes a cross-sectional view of the strut portion of FIG. 2B at line D-D.

FIG. 3A-3G shows perspective view of struts from certain embodiments, in which the struts comprise a cavity.

FIG. 4A-4M show perspective views of struts from additional embodiments, in which the struts comprise a cavity.

FIG. 5A-5G show cross-sectional views of struts from certain embodiments, in which the struts comprise a cavity.

FIG. 6 shows a perspective view of a strut of another embodiment.

FIG. 7 shows a perspective view of an embodiment of a stent in which a coating composition is disposed on a side surface of a strut of the stent.

FIG. 8A-8F show one embodiment of a method of making a stent.

FIG. 9 shows a step in an embodiment of a method of making a stent.

FIG. 10A-10F show another embodiment of a method of making a stent.

FIG. 11 shows a step in an embodiment of a method of making a stent.

FIG. 12A-12C show another embodiment of a method of making a stent.

FIG. 13A-13B show yet another embodiment of a method of making a stent.

### DETAILED DESCRIPTION

### The Medical Devices

In one embodiment, the medical device is a stent comprising a stent sidewall structure, which comprises a plurality of struts and gaps in the sidewall structure. At least one strut comprises a first material. Also, the at least one strut comprises an abluminal surface, a luminal surface, and a first side surface. The abluminal surface of the strut is the surface that faces away from the lumen or towards the lumen wall, e.g., a wessel wall, when the stent is implanted in a lumen. The luminal surface of the strut is the surface that faces toward the lumen or away from the lumen wall when the stent is implanted in a lumen. The side surface of the strut is a surface that is disposed between the abluminal and luminal surfaces of the strut. In some instances, where the strut is formed by being cut from a material, such as a metal tube, the side surface can be cut-surface, i.e. the surface that is formed when the strut is cut from the material. Furthermore, there is at least one cavity disposed in the at least one strut, wherein the cavity comprises an opening that is in fluid communication with a side surface of the strut. A therapeutic agent can be disposed in the cavity. This agent can be released from the cavity through the opening.

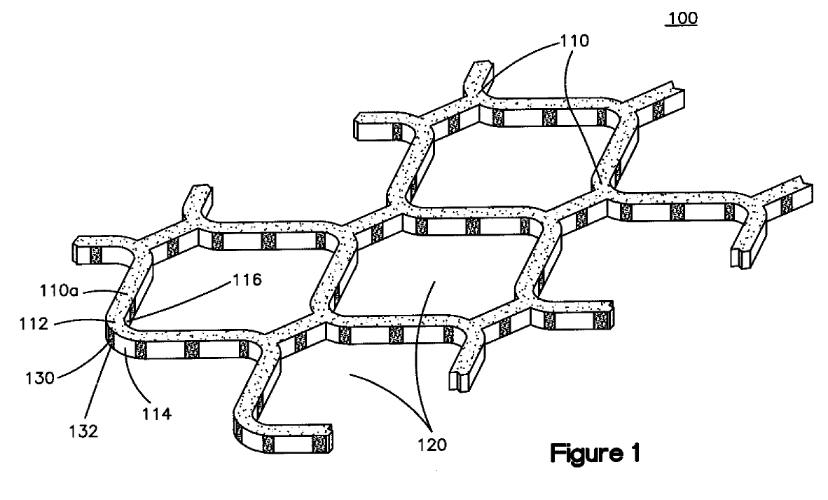

FIG. 1 shows an embodiment of a portion of a sidewall structure 100 of a stent. The sidewall structure 100 comprises struts 110 and gaps 120. At least one of the struts 110a has an abluminal surface 112 and a first side surface 114 and a second side surface 116. Also, the at least one strut 110a comprises a cavity 130 disposed therein. A therapeutic agent can be disposed in the cavity. The cavity 130 has an opening 132 that is in fluid communication with the first side surface 114. The cavity 130 can also have another opening (not shown) that is in fluid communication with the second side surface 116, the abluminal surface 112 or the luminal surface (not shown) of the strut 110a. In this embodiment, the opening 132 is at least partially exposed, i.e., not covered by a material. In some embodiments, the entire opening is exposed or a part of the opening is exposed.

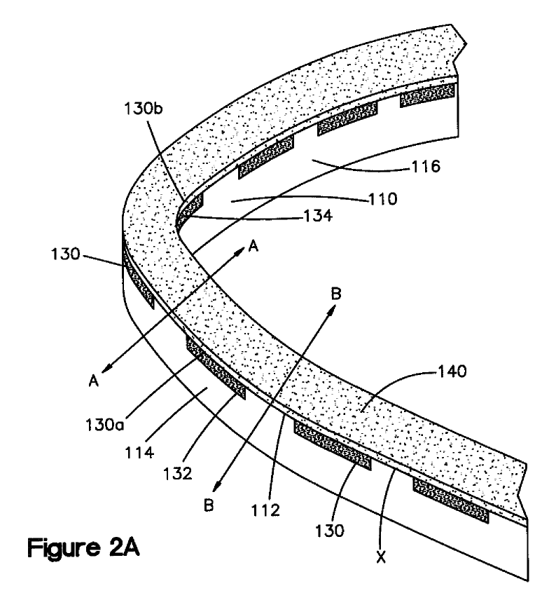

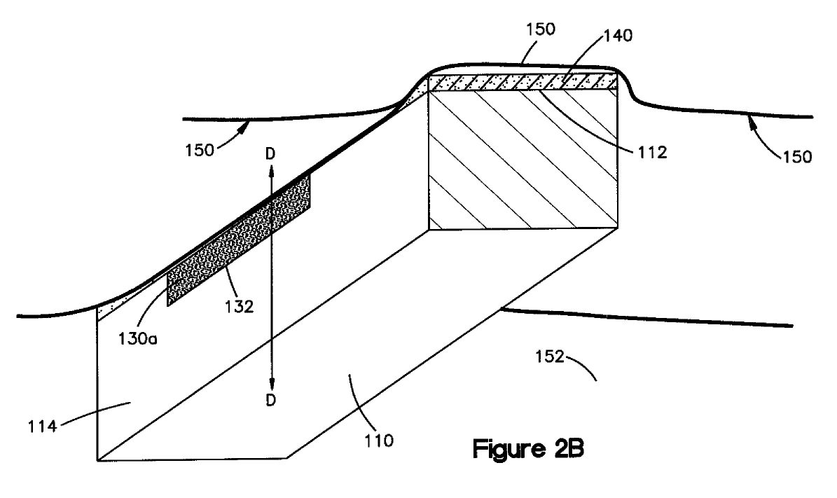

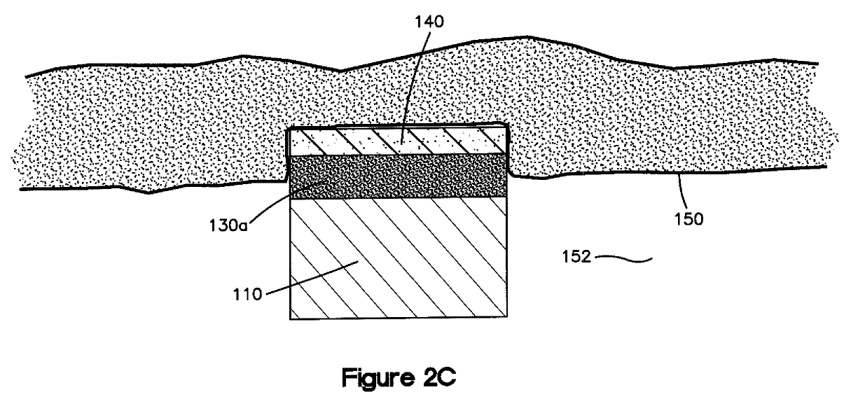

Shown is FIG. 2A is one embodiment of a strut. In this embodiment, a coating is disposed over an opening of a cavity that is disposed in the strut. More specifically, in this embodiment, the strut 110 has a number of cavities 130 disposed therein. At least one of the cavities 130a has a first opening 132 that is in fluid communication with a side surface 114 of the strut 110. The cavity 130a also has a second opening (not shown) that is in fluid communication with the abluminal surface 112 of the strut 110. Also, the cavity 130a can have a third opening (not shown) that is in fluid communication with the second side surface 116 of the strut 110, like opening 134 of cavity 130b. As described below, a cavity can have various configurations. Also, cavities having different configurations can be disposed in the same or different struts. Furthermore, as described below, the opening can have various shapes or configurations and opening of different shapes or configurations can be disposed in the same or different struts.

In this embodiment, a coating 140 is disposed over at least a portion of the opening of the cavity 130a that is in fluid communication with the abluminal surface 112. In some embodiments, the coating is disposed over the entire opening of the cavity that is in fluid communication with the abluminal surface. In others, the coating is disposed over less than the entire opening. In addition, in this embodiment, the coating 140 is disposed over a portion of the abluminal surface 112 of the strut 110, at for example position X.

The coating can comprise the same material as the strut. Alternatively, the coating can comprise a material that is different from the material of the strut. In certain embodiments, the coating comprises a polymer, a metal, an oxide, a ceramic, or different combination or composites of such materials (e.g., a composite of a ceramic and a polymer). In certain embodiments, the coating can comprise a radiopaque material.

In some instances, the coating is substantially free of any polymer, i.e., no polymer is intentionally added to the coating material. In other embodiments, the coating can comprise a polymer that modulates release of the therapeutic agent from the cavity through the coating. Also, in some embodiments, the coating comprises a material that prevents the release of the therapeutic agent from the cavity through the coating. In the alternative, the coating comprises a material have a plurality of pores therein that allows for the release of the therapeutic agent from the cavity through the coating. In the alternative, the coating comprises a material have a plurality of pores therein that allows for the release of the therapeutic agent from the cavity through the coating. Moreover, in come embodiments, the coating can comprise a therapeutic agent, which can be the same as or different from the therapeutic agent disposed in the cavity or cavities. For example, the coating can comprise a polymer and a first therapeutic agent. In such an embodiment, the first therapeutic agent is released from the abluminal surface of the strut while a second therapeutic agent in the cavity is released from the cut face.

FIG. 2B shows the portion of the strut 110 situated between lines A-A and B-B in FIG. 2A. The portion of the stent is shown as being inserted or implanted in a body lumen, such as a blood vessel 152. As shown in FIG. 2B, the coating 140 is placed in contact with the vessel wall 150. Since the opening 132 of the cavity 130a is at least partially exposed, the therapeutic agent disposed in the cavity 130a can be released from the cavity 130a into the vessel 152 or vessel wall 150. FIG. 2C shows a cross-sectional view of the strut shown in FIG. 2B at line D-D. As shown in this figure, the coating 140 is disposed over the opening of the cavity 130a that is in fluid communication with the abluminal surface of the strut 110. Also, as shown in this figure, the first and third openings that are each in fluid communication with a side surface are in contact with at least a portion of the second opening that is in fluid communication with the abluminial surface i.e., there is no material separating at least a portion of the opening.

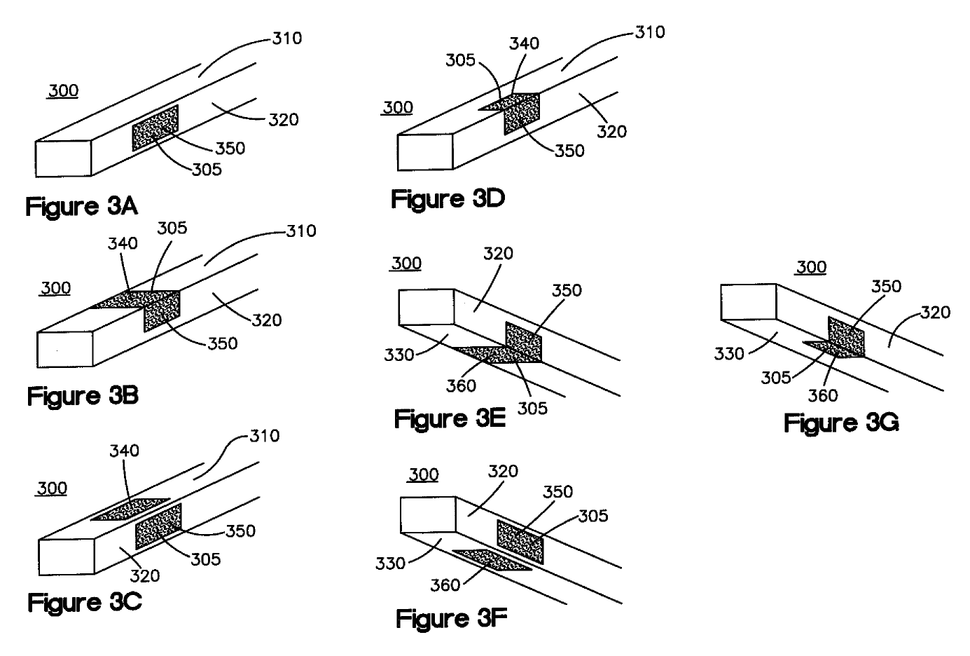

FIGS. 3A-3G show various configurations of openings of cavities 305 that can be disposed in a strut. Although these figures do not show a coating disposed over the opening(s) of the cavities, coatings can be disposed over at least a portion of one or more openings. FIG. 3A shows an example of a strut 300 having a cavity 305 disposed therein. The cavity 305 has an opening 350 at a single side surface 320. In this embodiment, the cavity 305 has no other opening that extends through a surface of the strut 300. In other embodiments, the single opening of the cavity can be present in a different surface of the strut, such as the abluminal surface 310.

FIGS. 3B, 3C and 3D show embodiments where the cavity 305 has a first opening 340 that is in fluid communication with the abluminal surface 310 of the strut 300 and a second opening 350 that is in fluid communication with a side surface 320 of the strut 300. In FIGS. 3B and 3D, a least a portion of opening 340 is in contact with a portion of opening 350, i.e. there is no material separating the portions of the openings. In FIG. 3 C, the openings 340, 350 are not in contact with each other. Furthermore, in FIG. 3B, the opening 340 extends across the entire width of the abluminal surface 310. In FIG. 3D, the opening 340 does not extend across the entire width of the abluminal surface 310. Also, in FIGS. 3B, 3C and 3D, the cavities may or may not have additional openings, like for instance in the luminal surface or other side surface (not shown).

A cavity 305 can also have an opening 360 that is in fluid communication with the luminal surface 330 of the strut 300 as shown in FIGS. 3E, 3F and 3G. In FIGS. 3E and 3G, a least a portion of opening 360 is in contact with a portion of opening 350. In FIG. 3F, the openings 350, 360 are not in contact with each other. Furthermore, in FIG. 3E, the opening 360 extends across the entire width of the luminal surface 330. In FIG. 3G, the opening 360 does not extend across the entire width of the luminal surface 330. Also, in FIGS. 3E, 3F and 3G, the cavities may or may not have additional openings, like for instance in the abluminal surface or other side surface (not shown).

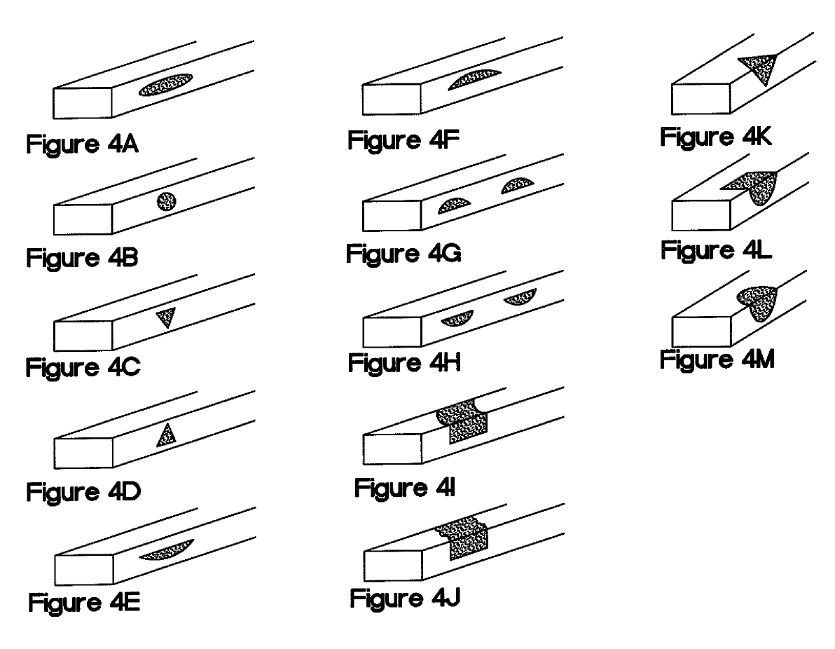

The openings of the cavities may have various shapes and sizes. FIGS. 4A-4M show examples of shapes of openings. FIG. 4A shows an opening having an oval shape, and FIG. 4B shows an opening having a circular shape. FIGS. 4C and 4D show openings having triangular shapes. FIGS. 4E and 4F show openings having half-oval shapes, and FIGS. 4G and 4H show openings having semicircular shapes. FIGS. 4I-4M show examples of opening having various shapes in which at least a portion of one opening is in contact with a portion of another opening. Various modifications to the shapes, in addition to those shown and described herein, will become apparent to those skilled in the art.

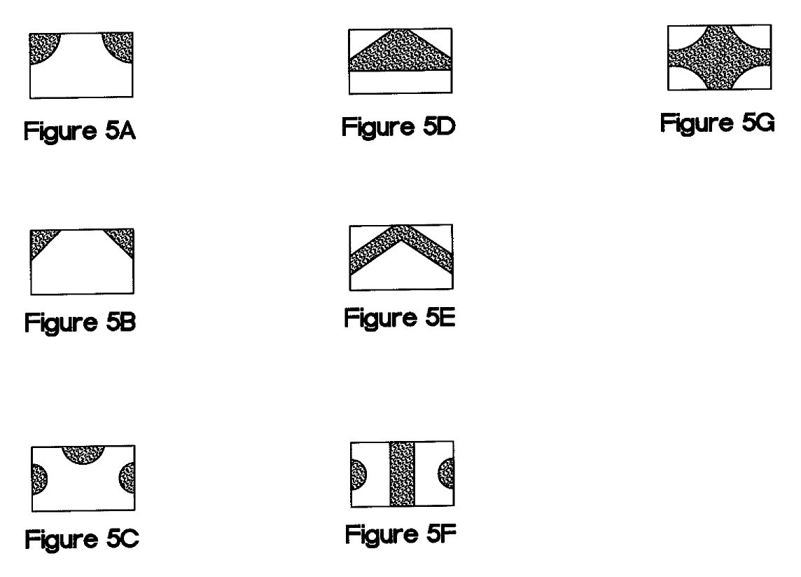

The cavities may have various shapes and sizes. FIGS. 5A to 5G show cross-sectional views of a number of cavity shapes. Various modifications to the shapes, in addition to those shown and described herein, will become apparent to those skilled in the art.

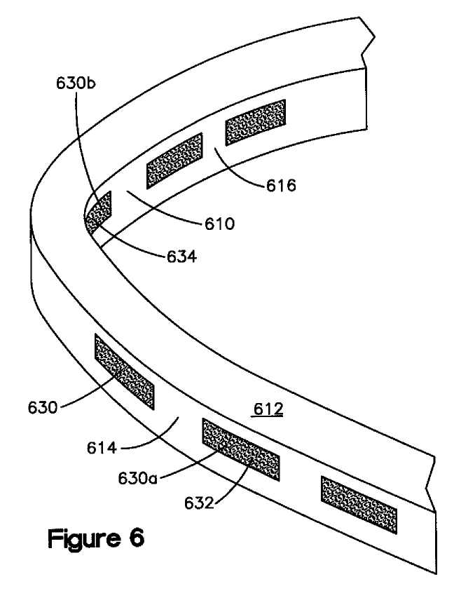

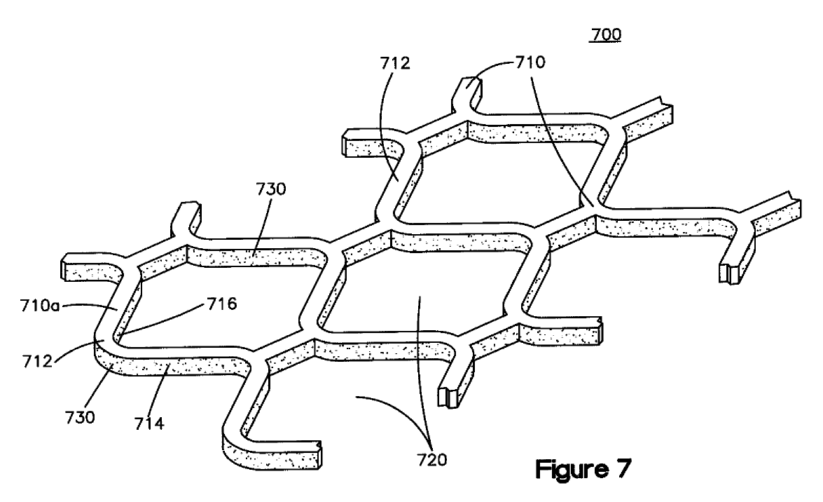

The embodiment shown in FIGS. 2A-2C includes a coating disposed over at least a portion of an opening of a cavity. Other embodiments of struts, such as any of those described above, may not include such a coating. FIG. 6 shows an embodiment that does not include such a coating. In this embodiment, the strut 610 has a number of cavities 630 disposed therein. At least one of the cavities 630a has an opening 632 that is in fluid communication with a side surface 614 of the strut 610. The cavity 630a also has an opening (not shown) that is in fluid communication with the other side surface 616 of the strut 610, like opening 634 of cavity 630b. In this embodiment, the openings of the cavities are at least partially exposed. In addition, in this embodiment, the cavity 630a does not have any openings that are in fluid communication with the abluminal surface 612 or the luminal surface of the strut 610. Thus, a therapeutic agent that is disposed in the cavity cannot be released through the abluminal or luminal surface of the strut. The therapeutic agent can be released through the openings in the side surfaces of the strut. In some embodiments, a coating can be disposed on one or more surfaces of the strut. The coating can comprise the materials discussed herein.

In yet another embodiment, the medical device comprises a stent having a sidewall structure 700 comprising a plurality of struts 710 and gaps 720 in the sidewall structure 700. In certain embodiments, the stent is self-expanding stent. At least one of the struts 710, such as strut 710a, has an abluminal surface 712, a luminal surface (not shown) and a first side surface 714 and a second side surface 716. A coating 730, which can comprise a polymer and/or a therapeutic agent, is disposed on at least a portion of one or more of the side surfaces 714, 716 of the strut 710. The abluminal surfaces 712 and luminal surfaces of the struts are substantially free of the coating, i.e., no coating is intentionally disposed on these surfaces. In some embodiments, the abluminal and luminal surfaces are substantially free of any coating.

### Types of Medical Devices

Medical devices suitable for the present embodiments, but are not limited to, those that have a tubular or cylindrical like portion. For example, the tubular portion of the medical device need not be completely cylindrical. The cross-section of the tubular portion can be any shape, such as rectangle, a triangle, etc., not just a circle. Such devices include, but are not limited to, stents, balloon catheters, and grafts. A bifurcated stent is also included among the medical devices which can be fabricated by the methods described herein.

In addition, the tubular portion of the medical device may be a sidewall that may comprise a plurality of struts defining a plurality of openings. The sidewall defines a lumen. The struts may be arranged in any suitable configuration. Also, the struts do not all have to have the same shape or geometric configuration. When the medical device is a stent comprising a plurality of struts, the surface is located on the struts. Each individual strut has an outer surface adapted for exposure to the body tissue of the patient, an inner surface, and at least one side surface between the outer surface and the inner surface.

Medical devices that are particularly suitable for the embodiments described herein include any kind of stent for medical purposes which is known to the skilled artisan. The stents can be intravascular stents that are designed for permanent implantation in a blood vessel of a patient. In certain embodiments, the stent comprises an open lattice sidewall stent structure. In exemplary embodiments, a stent suitable is a coronary stent. Other suitable stents include, for example, vascular stents such as self-expanding stents and balloon expandable stents. Examples of self-expanding stents that can be used are illustrated in U.S. Pat. Nos. 4,655,771 and 4,954,126 issued to Wallsten and U.S. Pat. No. 5,061,275 issued to Wallsten et al. Examples of appropriate balloon-expandable stents are shown in U.S. Pat. No. 5,449,373 issued to Pinchaski et al.

In one embodiments, the intravascular stent is generally cylindrical in shape. The stent includes a sidewall structure which comprises a plurality of struts and at least one gap in the sidewall structure. Generally, the gap is disposed between adjacent struts. Also, the sidewall structure may have a first sidewall surface and an opposing second sidewall surface. The first sidewall surface can be an outer or abluminal sidewall surface, which faces a body lumen wall when the stent is implanted, or an inner or luminal sidewall surface, which faces away from the body lumen surface. Likewise, the second sidewall surface can be an abluminal sidewall surface or a luminal sidewall surface. At least one strut comprises an abluminal surface, which forms part of the abluminal surface of the stent, and at least one strut comprises a luminal surface opposite the abluminal surface of the strut, which forms part of the luminal surface of the stent.

In some embodiments, the abluminal surface of the stent sidewall structure comprises at least on cavity and the luminal surface is free of cavities. In other embodiments, the cavity or cavities can be located on a low-stress bearing part of the stent sidewall structure.

When the coatings described herein are applied to a stent having openings in the stent sidewall structure, in certain embodiments, in come embodiments, the coatings conform to the surface of the stent so that the openings in the sidewall stent structure are preserved. e.g. the openings are not entirely or partially occluded with coating material.

The framework of suitable stents may be formed through various methods as known in the art. The framework may be welded, molded, laser cut, electro-formed, or consist of filaments or fibers which are wound or braided together in order to form a continuous structure.

The medical devices may be fabricated from a metallic material, ceramic material, polymeric or non-polymeric material, or a combination thereof (see Sections 5.1.1.1 to 5.1.1.3 infra.). Preferably, the materials are biocompatible. The material may be porous or non-porous, and the porous structural elements can be microporous or nanoporous. Further the coating may be a different material from the substrate or the same material as the substrate.

### Metallic Materials for Medical Devices

In certain embodiments, the medical device comprises a substrate or coating that is metallic. Suitable metallic materials useful for making the substrate or the coating include, but are not limited to, metals and alloys based on titanium (such as nitinol, nickel titanium alloys, thermo memory alloy materials), stainless steel, gold, platinum, iridium, molybdenum, niobium, palladium, chromium, tantalum, nickel, nickel chrome, cobalt, tungsten, or alloys thereof and/or combinations thereof. Examples of alloys include platinum iridium alloys, cobalt-chromium alloys, including cobalt chromium nickel alloys such as Elgiloy® and Phynox®, MP35N alloy, and nickel-titanium alloys, for example, Nitinol. Other metallic materials that can be used to make the medical device include clad composite filaments, such as those disclosed in WO 94/16646.

In some embodiments, the metal is a radiopaque material that makes the medical device visible under X-ray or fluoroscopy. Suitable materials that re radiopaque include, but are not limited to, gold, tantalum, platinum, bismuth, iridium, zirconium, iodine, titanium, barium, silver, tin, alloys of these metals, or a combination thereof.

Furthermore, although a single type of metal can be used to form the substrate, various combinations of metals can also be employed. The appropriate mixture of metals can be coordinated to produce desired effects when incorporated into a substrate.

### Ceramic Materials for Medical Devices

In certain embodiments, the medical device comprises a substrate or a coating which is ceramic. Suitable ceramic materials used for making the substrate or coating include, but are not limited to, oxides, carbides, or nitrides of the transition elements such as those containing titanium, hafnium, iridium, chromium, aluminum, zirconium, transition metals, platinum, tantalum, niobium, tungsten, rhodium, iron, vanadium, nickel, or a combination thereof. Silicon based materials, such as silica, may also be used. Furthermore, although a single type of ceramic can be used to form the substrate, various combinations of ceramics can also be employed. The appropriate mixture of ceramics can be coordinated to produce desired effects when incorporated into a substrate.

### Polymeric Materials for Medical Devices

In certain embodiments, the medical device comprises a substrate or a coating which is polymeric. In other embodiments, the material can be a non-polymeric material. The polymer(s) useful for forming the components of the medical devices should be ones that biocompatible and avoid irritation to body tissue. The polymers can be biostable or bioabsorbable. Suitable polymeric materials useful for making the substrate include, but are not limited to, isobutylene-based polymers, polystyrene-based polymers, polyacrylates, and polyacrylate derivatives, vinyl acetate-based polymers and its copolymers, polyurethane and its copolymers, silicone and its copolymers, ethylene vinyl-acetate, polyethylene terephtalate, thermoplastic elastomers, polyvinyl chloride, polyolefins, cellulosics, polyamides, polyesters, polysulfones, polytetrafluorethylenes, polycarbonates, acrylonitrile butadiene styrene copolymers, acrylics, polylactic acid, polyglycolic acid, polycaprolactone, polylactic acid-polyethylene oxide copolymers, cellulose, collagens, chitins, or a combination thereof.

Other polymers that are useful as materials for making the substrate include, but are not limited to, Dacron polyester, poly(ethylene terephthalate), polycarbonate, polymethylmethacrylate, polypropylene, polyalkylene oxalates, polyvinylchloride, polyurethanes, polysiloxanes, nylons, poly(dimethyl siloxane), polycyanoacrylates, polyphosphazenes, poly(amino acids), ethylene glycol I dimethacrylate, poly(methyl methacrylate), poly(2-hydroxyethyl methacrylate), polytetrafluoroethylene poly(HEMA), polyhydroxyalkanoates, polytetrafluorethylene, polycarbonate, poly(glycolide-lactide) co-polymer, polylactic acid, poly(ε-caprolactone), poly(β-hydroxybutyrate), polydioxanone, poly(γ-ethyl glutamate), polyiminocarbonates, poly(ortho ester), polyanhydrides, styrene isobutylene styrene, polyetheroxides, polyvinyl alcohol, polyglycolic acid, polylactic acid, polyamides, poly-2-hydroxy-butyrate, polycaprolactone, poly(lactic-co-clycolic)acid, Teflon, alginate, dextran, chitin, cotton, polyglycolic acid, polyurethane, derivatized versions thereof, (i.e., polymers which have been modified to include, for example, attachment sites or cross-linking groups, e.g. arginine-glycine-aspartic acid RGD, in which the polymers retain their structural integrity while allowing for attachment of cells and molecules, such as proteins and/or nucleic acids), or a combination thereof.

The polymers may be dried to increase their mechanical strength. The polymers may then be used as the base material to form a whole or part of the substrate.

Furthermore, although a single type of polymer can be used to form the substrate, various combinations of polymers can also be employed. The appropriate mixture of polymers can be coordinated to produce desired effects when incorporated into a substrate.

### Therapeutic Agents

The term “therapeutic agent” as used herein encompasses drugs, genetic materials, and biological materials and can be used interchangeably with “biologically active material”. The term “genetic material” means DNA or RNA, including, without limitation, DNA/RNA encoding a useful protein stated below, intended to be inserted into a human body including viral vectors and non-viral vectors.

The term “biological materials” include cells, yeasts, bacterial, proteins, peptides, cytokines and hormones. Examples for peptides and proteins include vascular endothelial growth factor (VEGF), transforming growth factor (TGF), fibroblast growth factor (FGF), epidermal growth factor (EGF), cartilage growth factor (CGF), nerve growth factor (NGF), keratinocyte growth factor (KGF), skeletal growth factor (SGF), osteoblast-derived growth factor (BDGF), hepatocyte growth factor (HGF), insulin-like growth factor (IGF), cytokine growth factors (CGF), platelet-derived growth factor (PDGF), hypoxia inducible factor-1 (HIF-1), stem cell derived factor (SDF), stem cell factor (SCF), endothelial cell growth supplement (ECGS), granulocyte macrophage colony stimulating factor (GM-CSF), growth differentiation factor (GDF), integrin modulating factor (IMF), calmodulin (CaM), thymidine kinase (TK), tumor necrosis factor (TNF), growth hormone (GH), bone morphogenic protein (BMP) (e.g., BMP-2, BMP-3, BMP-4, BMP-5, BMP-6 (Vgr-1), BMP-7 (PO-1), BMP-8, BMP-9, BMP-10, BMP-11, BMP-12, BMP-14, BMP-15, BMP-16, etc.), matrix metalloproteinase (MMP), tissue inhibitor of matrix metalloproteinase (TIMP), cytokines, interleukin (e.g., IL-1, IL-2, IL-3, IL-4, IL-5, IL-6, IL-7, IL-8, IL-9, IL-10, IL-11, IL-12, IL-15, etc.), lymphokines, interferon, integrin, collagen (all types), elastin, fibrillins, fibronectin, vitronectin, laminin, glycosaminoglycans, proteoglycans, transferring, cytotactin, cell binding domains (e.g., RGD), and tenascin. Examplary BMP's are BMP-2, BMP-3, BMP-4, BMP-5, BMP-6, BMP-7. These dimeric proteins can be provided as homodimers, heterodimers, or combinations thereof, alone or together with other molecules. Cells can be of human origin (autologous or allogeneic) or from an animal source (xenogeneic), genetically engineered, if desired, to deliver proteins of interest at the transplant site. The delivery media can be formulated as needed to maintain cell function and viability. Cells include progenitor cells (e.g., endothelial progenitor cells), stem cells (e.g., mesenchymal, hematopoietic, neuronal), stromal cells, parenchymal cells, undifferentiated cells, fibroblasts, macrophage, and satellite cells.

Other suitable therapeutic agents include:
anti-thrombogenic agents such as heparin, heparin derivatives, urokinase, and PPack (dextrophenylalanine praline arginine chloromethylketone);
anti-proliferative agents such as enoxaprin, angiopeptin, or monoclonal antibodies capable of blocking smooth muscle cell proliferation, hirudin, acetylsalicylic acid, tacrolimus, everolimus, pimecrolimus, sirolimus, zotarolimus, amlodipine and doxazosin;
anti-inflammatory agents such as glucorticoids, betemethasone, dexamethasone, prednisolone, corticosterone, budesonide, estrogen, sulfasalazine, rosiglitazone, mycophenolic acid and mesalamine;
anti-neoplastic/anti-proliferative/anti-miotic agents such as paclitaxel, 5-fluorouracil, cisplatin, vinblastine, vincristine, epothilones, methotrexate, azathioprine, adriamycin and mutamycin; endostatin, angiostatin and thymidine kinase inhibitors, cladribine, taxol and its analogs or derivatives, paclitaxel as well as its derivatives, analogs or paclitaxel bound to proteins, e.g. Abraxane™;
anesthetic agents such as lidocaine, bupivacaine, and ropivacaine;
anit-coagulants such as D-Phe-Pro-Arg chloromethyl ketone, an RGD peptide-containing compound, heparin, antithrombin compounds, platelet receptor antoagonists, anti-thrombin antibodies, anti-platelet receptor antibodies, aspirin (aspirin is also classified as an analgesic, antipyretic and anti-inflammatory drug), dipyridamole, protamine, hirudin, prostaglandin inhibitors, platelet inhibitors, antiplatelet agents such as trapidil or liprostin and tick antiplatelet peptides;
DNA demethylating drugs such as 5-azacytidine, which is also categorized as a RNA or DNA metabolite that inhibit cell growth and induce apoptosis in certain cancer cells;
vascular cell growth promoters such as growth factors, vascular endothelial growth factors (VEGF, all types including VEGF-2), growth factor receptors, transcriptional activators, and translational promoters;
vascular growth inhibitors such as anti-proliferative agents, growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin;
cholesterol-lowering agents, vasodilating agents, and agents which interfere with endogenous vasoactive mechanisms;
anti-oxidants, such as probucol;
antibiotic agents, such as penicillin, cefoxitin, oxacillin, tobranycin, rapamycin (sirolimus);
angiogenic substances, such as acidic and basic fibroblast growth factors, estrogen including estradiol (E2), estriol (E3) and 17-beta estradiol;
drugs for heart failure, such as digoxin, beta-blockers, angiotensin-convertin enzyme (ACE) inhibitors including captropril and enalopril, statins and related compounds; and
macrolides such as sirolimus or everolimus;

Other therapeutic agents include nitroglycerin, nitrous oxides, nitric oxides, antibiotics, aspirins, digitalis, estrogen, estradiol and glycosides. Exemplary therapeutic agents include anti-proliferative drugs such as steroids, vitamins, and restenosis-inhibiting agents. Exemplary restonosis-inhibiting agents include microtubule stabilizing agents such as Taxol®, paclitaxel (i.e., paclitaxel, paxlitaxel analogs, or paclitaxel derivatives, and mixtures thereof). For example, derivatives suitable for use in the medical devices include 2′-succinyl-taxol, 2′-succinyl-taxol triethanolamine, 2′-glutaryl-taxol, 2′glutaryl-taxol triethanolamine salt, 2′-O-ester with N-(dimethylaminoethyl)glutamine, and 2′-O-ester with N-(dimethylaminoethyl)glutamide hydrochloride salt.

Other exemplary therapeutic agents include tacrolimus; halafuginone; inhibitors of HSP90 heart shock proteins such as geldanamysin; microtubule stabilizing agents such as epothilone D; phosphodiesterase inhibitors such as cliostazole; Barkct inhibitors; phospholamban inhibitors; and Serca 2 gene/proteins. In yet another embodiment, the therapeutic agent is an antibiotic such as erythromycin, amphotericin, rapamycin, adriamycin, etc.

In one embodiment, the therapeutic agent is capable of altering the cellular metabolism or inhibiting a cell activity, such as protein synthesis, DNA synthesis, spindle fiber formation, cellular proliferation, cell migration, microtubule formation, microfilament formation, extracellular matrix synthesis, extracellular matrix secretion, or increase in cell volume. In another embodiment, the therapeutic agent is capable of inhibiting cell proliferation and/or migration.

In certain embodiments, the therapeutic agents for use in the medical devices can be synthesized by methods well known to one skilled in the art. Alternatively, the therapeutic agents can be purchased from chemical and pharmaceutical companies.

### Methods of Making the Medical Devices

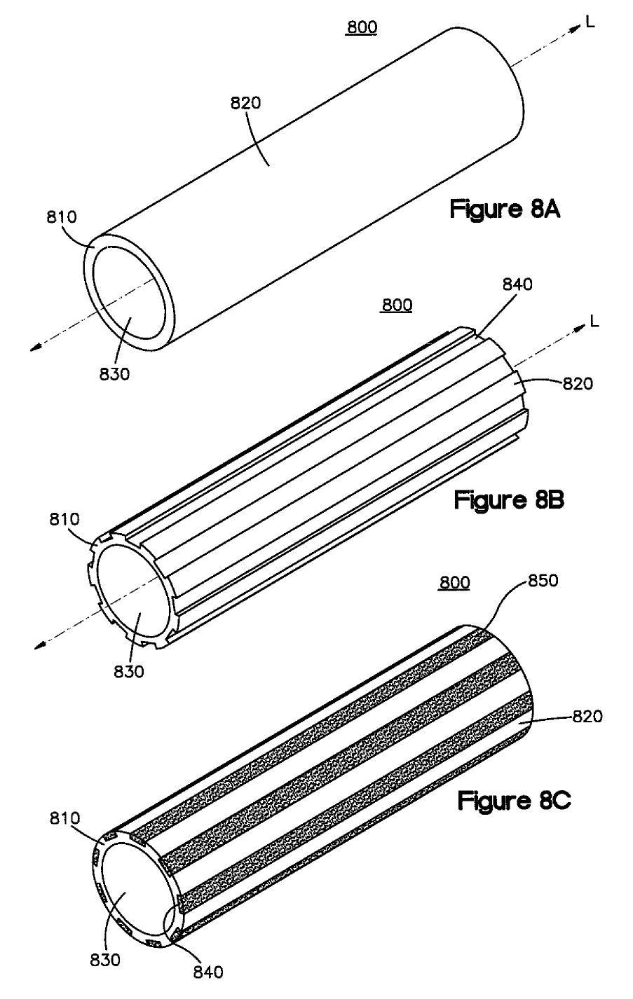

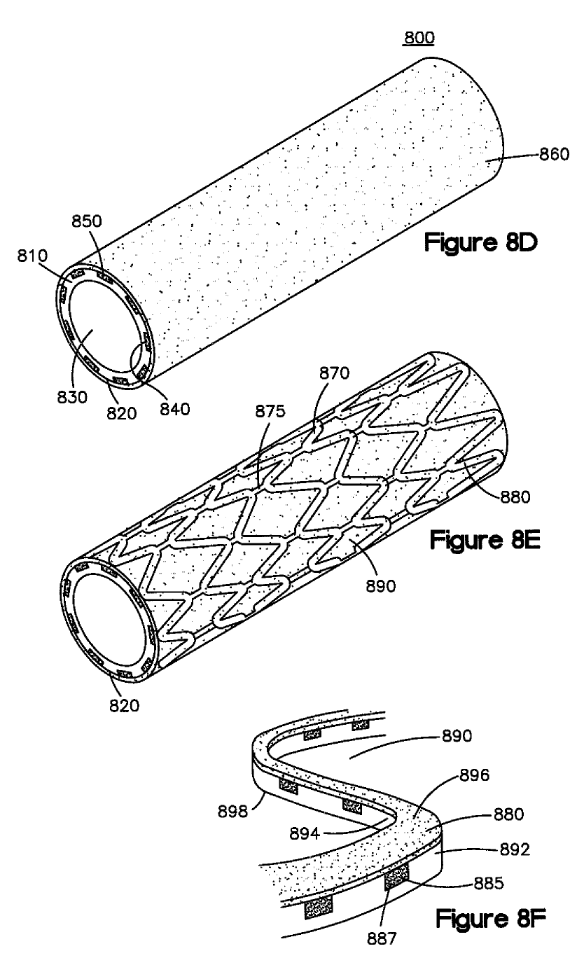

Provided herein are methods of making the medical devices described herein. FIGS. 8A-8F show one embodiment of a method for making an implantable stent. This method comprises providing a tube 800 having a tubular wall 810 and a longitudinal axis L as shown in FIG. 8A. The tubular wall 810 comprises an abluminal surface 820 and a luminal surface 830. As shown in FIG. 8B, at least one groove 840 is disposed in the abluminal surface 820 of the tubular wall 810. The grooves 840 do not extend through the tubular wall 810 to the luminal surface 830. In come embodiments, the method can include the step of forming the groove(s) in the abluminal surface.

The grooves can be formed by various methods, which include without limitation grinding, scoring, or using a laser to remove tube material. Also, the grooves can be formed by any other method known to one skilled in the art, including, but not limited to, sintering, co-deposition, micro-roughing, drilling, chemical etching or a combination thereof. For example, the grooves can be made by a deposition process such as sputtering with adjustments to the deposition condition, by micro-roughening using reactive plasmas, by ion bombardment electrolyte etching, or a combination thereof. Other methods include, but are not limited to, alloy plating, physical vapor deposition, chemical vapor deposition, sintering, or a combination thereof. In addition to material removal techniques, other methods may include incorporating protrusions into a mold used by one skilled in the art for forging the tube or stent. The protrusions create grooves or cavities in the forge tube or stent. Other methods include extruding a tube with grooves already incorporated into the tube.

In the embodiment shown in FIG. 8B, the grooves are formed such that they are parallel to the longitudinal axis L of the tube 800. In other embodiments, the grooves can have other configurations such as being circumferential or parallel to the circumference of the tube, i.e., perpendicular to the longitudinal axis, being in the shapes of a helix, or in some other desired shape or pattern. The grooves can also be intermittent, i.e., not is a regular pattern or not continuing through the entire tube. The grooves can be of an intermittent longitudinal configuration and/or an intermittent circumferential configuration. The grooves can be intermittent in a configuration such that, upon cutting the stent pattern from the tube, the grooves cross the stent struts. This crossing can be perpendicular to the stent strut.

As shown in FIG. 8C, a therapeutic agent 850 is disposed in at least a portion of a groove 840. In some embodiments, such as the one shown in FIG. 8C, the therapeutic agent is disposed in the entire groove. In other embodiments, the therapeutic agent is disposed in less that the entire groove. Such therapeutic agent can include, but are not limited to, any of the therapeutic agents described herein. The therapeutic agent can be disposed in the groove by a micropen process, or a process involving masking and spraying, dipping, roll-cutting, or vapor deposition. The therapeutic agent can also be deposited by a bulk process, such as spray-coating, dip-coating, roll-coating, or vapor deposition, with the agent then removed from the non-grooved surfaces.

As shown in FIG. 8D, a coating 860 is disposed over at least a portion of the abluminal surface 820 of the tubular wall 810 after the therapeutic agent 850 is disposed in the groove 840, such that at least a portion of the therapeutic agent 850 disposed in the groove 840 is covered by the coating 860. In some embodiments, all of the therapeutic agent can be covered by the coating while in others less than all the therapeutic agent is covered. In the embodiment shown in FIG. 8D, the coating 860 is disposed over the entire abluminal surface 820 of the tubular wall 810. In other embodiments, the coating is disposed over less than the entire abluminal surface of the tubular wall.

The coating may comprise any of the materials listed herein. In certain embodiments, the coating comprises a material that prevents the release of the therapeutic agent through the coating. In other embodiments, the coating comprises a material having a plurality of pores therein that allows for the release of the therapeutic agent through the coating.

Also, the coating can be disposed on the abluminal surface by methods such as spray-coating, dip-coating, roll coating, or vapor deposition.

As shown in FIG. 8E, a stent 870 is formed from the tube 800, having the coating 860 disposed on the abluminal surface 820. The stent 870 has a stent sidewall structure 875 comprising a plurality of struts 880 and gaps 890. As shown in FIG. 8F, at least one of the struts 880 comprises an abluminal surface 896, a luminal surface 898, a first side surface 892, a second side surface 894 and a cavity 885. The cavity 885 comprises a portion of the groove 840 containing the therapeutic agent 850, and an opening 887 that is in fluid communication with the first side surface 892 and/or second side surface 894. The opening 887 is at least partially exposed. The stent can be formed by cutting the stent sidewall structure from the tube. In certain embodiments, the cutting can be conducted by using a laser and/or by the use of masking and chemical etching.

Another embodiment of a method for making an implantable stent is similar to the one described above. However, instead of disposing a therapeutic agent in the groove, a filler material is disposed in at least a portion of the groove. After or during the formation of the stent from the tube, at least a portion of the filler material is removed from the cavity of the strut. The filler material can be removed by dissolving the filler material in a solvent. For instance, in one embodiment, the filler material can be dissolved by submerging the stent in a solvent. After at least a port of the filler material is removed, a therapeutic agent is then disposed in the cavity.

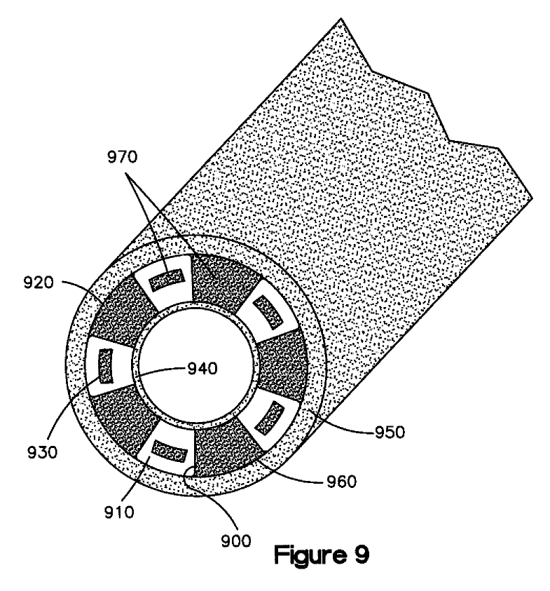

The therapeutic agent is disposed in the cavity in one embodiment by using a mandrel and a tube having an inner wall. In particular, the stent that is formed from the tube, is disposed on an expandable mandrel. The stent disposed on the mandrel is placed in a tube having an inner wall. The mandrel is expanded so that at least a portion of the stent contacts the inner wall of the tube. The stent is then exposed to a therapeutic agent and the therapeutic is allowed to enter the cavity. FIG. 9 shows a cross-sectional view of a stent 900 having struts 910 and gaps 920 disposed between a mandrel 940 and a tube 950 having an inner surface 960. The struts 910 have cavities 930 therein. In this figure, the stent 900 is exposed to a therapeutic agent 970, which has entered the cavities 930 and the gaps 920.

In an alternative embodiment, the stent is disposed on a rigid mandrel. The stent disposed on the mandrel is placed in a tube having an inner wall. Instead of expanding the mandrel, the tube is moved towards or closed around the stent so that at least a portion of the stent contacts the inner wall of the tube. The stent is then exposed to a therapeutic agent and the therapeutic agent is allowed to enter the cavity.

In some instances, the therapeutic agent is contained in a composition that is capable of becoming solid and the stent is exposed to such composition and the composition is allowed to enter the cavity. The composition can be allowed to enter by being pressure forced into the cavity. Also, in some embodiment, the method comprises the step of allowing the composition to solidify or harden.

Furthermore, in some embodiments, the method further comprises removing any excess therapeutic agent from the stent. Such excess therapeutic agent can be removed by laser-cutting, mechanical cutting or water-jet cutting.

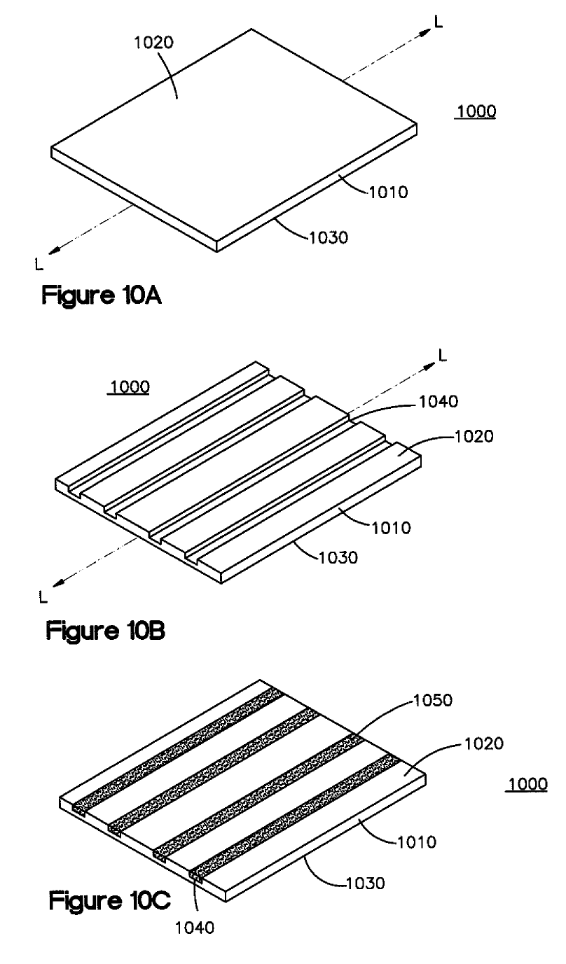

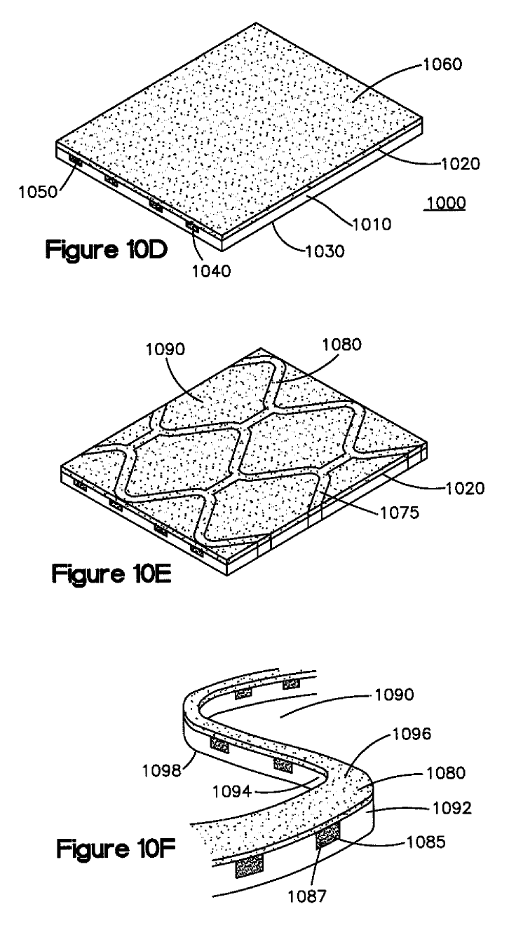

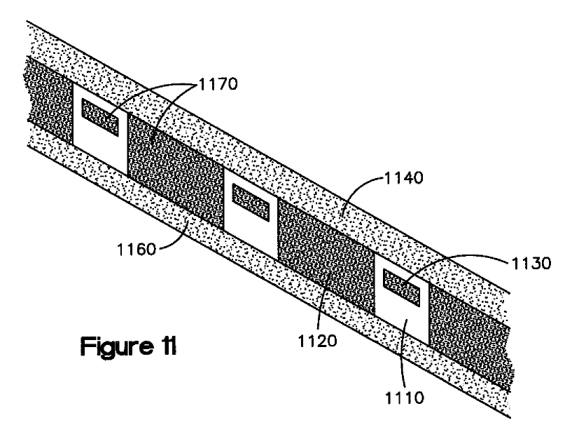

Another embodiment of a method is shown in FIGS. 10A-10F. This method comprises providing a flat sheet 1000 of a material having a sheet wall of 1010 and a longitudinal axis of L as shown in FIG. 10A. The sheet wall 1010 comprises a top or first surface 1020 and a bottom or second surface 1030. As shown in FIG. 10B, at least on e groove 1040 is disposed in the first surface 1020 of the tubular wall 1010. The grooves 1040 do not extend through the sheet wall 1010 to the second surface 1030. In some embodiments, the method can include the step of forming the groove(s) in the first surface. The grooves can be formed by various methods, including the methods stated herein.

In the embodiment shown in FIG. 10B, the grooves 1040 are formed such that they are parallel to the longitudinal axis L of the sheet 1000. In other embodiments, the grooves can have other configurations such as being perpendicular to the longitudinal axis, forming a checkered pattern on the first surface 1020, or in some other desired shape, pattern, or configuration such as those stated herein.

As shown in FIG. 10C, a therapeutic agent 1050 is disposed in at least a portion of a groove 1040. In some embodiments, such as the one shown in FIG. 10C, the therapeutic agent is disposed in the entire groove. In other embodiments, the therapeutic agent is disposed in the less than the entire groove. Such therapeutic agent can include, but are not limited to, any of the therapeutic agents described herein. The therapeutic agent can be disposed in the groove by processes such as those described herein.

As shown in FIG. 10D, a coating 1060 is disposed over at least a portion of the first surface 1020 of the sheet wall 1010 after the therapeutic agent 1050 is disposed in the groove 1040, such that at least a portion of the therapeutic agent 1050 disposed in the groove 1040 is covered by the coating 1060. In come embodiments, all of the therapeutic agent can be covered by the coating while in others less than all the therapeutic agent is covered. As shown in FIG. 10D, the coating 1060 is disposed over the entire first surface 1020 of the sheet wall 1010. In other embodiments, the coating is disposed over less than the entire first surface of the sheet wall. The coating may comprise any other material listed herein. Also, the coating can be disposed on the first surface by methods described herein.

As shown in FIG. 10E a sidewall structure 1075 is formed from the sheet 1000, having the coating 1060 disposed on the first surface 1020. After certain portions of the coated sheet are removed, the sidewall structure 1075 comprises a plurality of struts 1080 and gaps 1090 is formed. The sidewall structure can be formed by cutting the sidewall structure from the coated sheet. In certain embodiments, the cutting can be conducted by using a laser and/or by the use of masking and chemical etching.

As shown in FIG. 10F, at least one of the struts 1080 of the sidewall structure comprises a top surface 1096, which can be an abluminal or luminal surface, a bottom surface 1098, a first side surface 1092, a second side surface 1094 and a cavity 1085. The cavity 1085 comprise a portion of the groove 1040 containing the therapeutic agent 1050, and an opening 1087 that is in fluid communication with the first side surface 1092 and/or second side surface 1094. The opening 1087 is at least partially exposed. To obtain a tubular shape for a stent, the sheet or sidewall structure can be formed into a tubular shape at any time during the process. In some embodiments, the sidewall structure can be formed into a tubular shape before or after the sidewall structure is formed from the sheet.

Another embodiment of a method for making an implantable stent is similar to the one described above in connection with FIGS. 10A-10F. However, instead of disposing a therapeutic agent in the groove, a filler material is disposed in at least a portion of the groove. After or during the formation of the sidewall structure from the sheet, at least a portion of the filler material is removed from the cavity of the strut. The filler material can be removed by methods such as those described above in connection with FIG. 9. After at least a port of the filler material is removed, a therapeutic agent is then disposed in the cavity.

In addition, the therapeutic agent can be disposed in the cavity in one embodiment by using two flat surfaces. In particular, the sidewall structure that is formed from the sheet, is disposed between two flat surfaces. The sidewall structure is then exposed to a therapeutic agent and the therapeutic is allowed to enter the cavity. FIG. 11 shows a cross-sectional view of a sidewall structure having struts 1110 and gaps 1120 disposed between a first plate 1140 and a second plate 1160. The struts 1110 have cavities 1130 therein. In this figure, the sidewall structure is exposed to a therapeutic agent 1170, which has entered the cavities 1130 and the gaps 1120. The first plate 1140 and the second plate 1160 are shown as examples of items that can sandwich the sidewall structure between two surfaces. Various other items, in addition to the plates shown and described herein, will become apparent to those skilled in the art. Also, the sheet or sidewall structure can be formed into a tubular shape at any time during the process.

In another embodiment of a method for making an implantable stent, a therapeutic agent is disposed in the cavity of a strut of a stent after the strut has been formed. The method comprises providing a stent having a stent sidewall structure comprising a plurality of struts and gaps in the sidewall structure. At least one strut comprises an abluminal surface, a luminal surface, a first side surface, and at least one cavity disposed in the strut. The cavity has a first opening that is in fluid communication with the first side surface and second opening that is in fluid communication with the abluminal surface or the luminal surface. A therapeutic agent is disposed in the cavity. Thereafter, a material is applied over the second opening and any other opening so that the cavity is not in fluid communication with the abluminal surface or luminal surface. The first opening which is in fluid communication with the side surface, remains at least partially exposed.

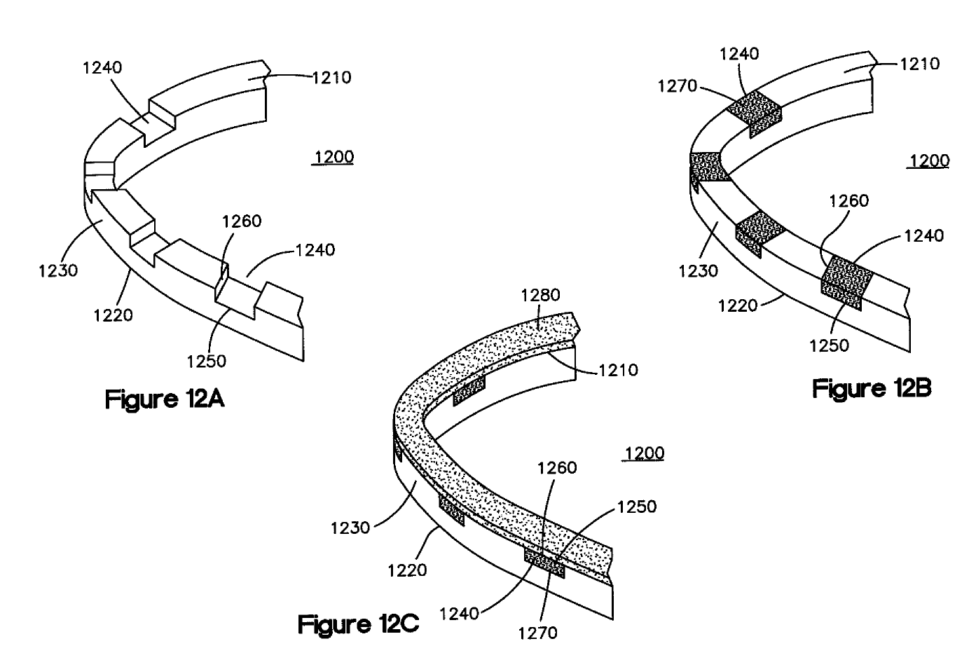

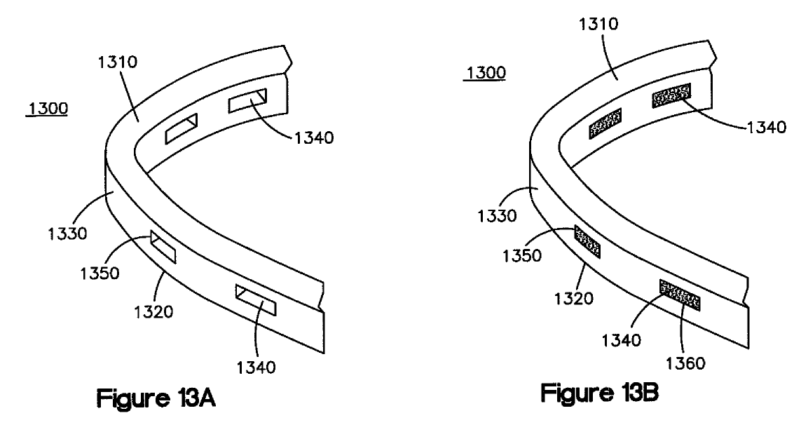

FIGS. 12A-12C show an example of such an embodiment. FIG. 12A shows a strut 1200 of a stent having an abluminal surface 1210, luminal surface 1220, and a first side surface 1230. The strut comprises at least one cavity 1240 having a first opening 1250 that is in fluid communication with the first side surface 1230 and a second opening 1260 that is in fluid communication with the abluminal surface 1210. At least a portion of the first opening is in contact with at least a portion of the second opening.

In FIG. 12B, a therapeutic agent 1270 is disposed in the cavities 1240. A material 1280, which can be the same as or different from the material from which the strut is made, is applied over the second opening 1260 of the cavities 1240, as shown in FIG. 12C. This material 1280 or another material is applied over any other opening of the cavities so that the cavities 1240 are not in fluid communication with the abluminal or luminal surface. The material can comprise those described above in connection with the coating described in FIG. 8D. As shown in FIG. 12C, the first opening 1250 is at least partially exposed.

In addition, the method can further comprise forming the stent sidewall structure by providing a tube having a tubular wall and a longitudinal axis, in which the tubular wall comprises an abluminal surface and a luminal surface, such as the one shown in FIG. 8A. There is at least one groove disposed in the abluminal surface of the tubular wall. The groove does not extend through the tubular wall to the luminal surface. FIG. 8B shows an example of such a tube having at least one groove. The stent sidewall structure is formed from the tube, such as by cutting the stent sidewall structure from the tube. The struts of the stent sidewall structure will comprise a cavity, which is a portion of the groove.

The method can further comprise forming the stent sidewall structure by providing a flat sheet having a sheet wall and longitudinal axis, in which the sheet wall comprises a first surface and second surface, such as the one shown in FIG. 10A. There is at least one groove disposed in the first surface of the sheet wall. The groove does not extend through the sheet wall to the second surface. FIG. 10B shows an example of such a sheet wall having at least one groove. A sidewall structure is formed from the flat sheet, such as by cutting the sidewall structure from the flat sheet. The struts of the sidewall structure will comprise a cavity, which is a portion of the groove. The flat sheet can be then formed into a tubular shape.

Moreover, another method for making an implantable stent, in which a therapeutic agent is disposed in the cavity of a strut after the strut has been formed, comprises providing a stent having a stent sidewall structure comprising a plurality of struts and gaps in the sidewall structure. FIG. 13A shows an example of a strut 1300 from such a stent. The strut 1300 comprises an abluminal surface 1310, a luminal surface 1320, a first side surface 1330, and at least one cavity 1340 disposed in the strut 1300. Each of the cavities 1340 has a first opening 1350 that is in fluid communication with the first side surface 1330 and at least one of the cavities 1340 is not in fluid communication with the abluminal surface 1310 or the luminal surface 1320. In FIG. 13B, a therapeutic agent 1360 is disposed in the cavities 1340; and the first opening 1350 are allowed to be exposed. In certain embodiments, the formation of the cavities can be conducted by using a laser-cutting or laser-ablation. In certain embodiments, the therapeutic agent is disposed in the cavity by disposing the stent on a mandrel. The stent disposed on the mandrel is placed in a tube having an inner wall. The tube is moved towards or closed around the stent so that at least a portion of the stent contacts the inner wall of the tube. The stent is then exposed to a therapeutic agent and the therapeutic agent is allowed to enter the cavity.

Another embodiment of a method for making an implantable stent comprises disposing a coating on at least one side surface of a stent strut while keeping the abluminal and luminal surfaces of the strut substantially free of the coating composition. This method comprises providing a stent having a stent sidewall structure comprising a plurality of struts and gaps in the sidewall structure, wherein at least one strut comprises an abluminal surface, a luminal surface, and a first side surface. The stent is disposed on a mandrel. A coating composition comprising a therapeutic agent is applied onto a flat surface. The stent disposed on the mandrel is rolled in the coating composition applied to the flat surface to apply the coating composition to the first side surface of the strut.

Steps are taken to ensure that the abluminal surface and the luminal surface are substantially free of the soating composition. In some embodiments, the stent is rolled with sufficient pressure such that the coating composition is applied to the first side surface while the abluminal surface is made substantially free of the coating composition. In other embodiments, the abluminal surface and luminal surface are made substantially free of the coating composition by removing any coating composition disposed on the abluminal surface or luminal surface after the coating composition is applied to the first side surface. The coating composition can be removed by mechanical grinding, laser-ablation, masking and etching, or chemical dissolution of the coating.

The therapeutic agent of the coating composition can be, but are not limited to, those described herein. Also, the coating composition can comprise a polymer, such as but not limited to those described above for forming medical devices. Furthermore, the coating composition can include a solvent for suspending or dissolving the therapeutic agent and/or polymer.

Some exemplary embodiments of medical devices and methods for making same in accordance with the present invention are described in the following numbered paragraphs:

Paragraph 1. A method for making an implantable stent comprising:

a. providing a stent having a stent sidewall structure comprising a plurality of struts and gaps in the sidewall structure, wherein at least one strut comprises an abluminal surface, a luminal surface, a first side surface, and at least one cavity disposed in the strut, wherein the cavity has a first opening that is in fluid communication with the first side surface and wherein the cavity is not in fluid communication with the abluminal surface or the luminal surface;

b. disposing a therapeutic agent in the cavity; and

c. allowing the first opening to be at least partially exposed.

Paragraph 2. A method for making an implantable stent comprising:

a. providing a flat sheet of a material having a sheet wall, in which the sheet wall comprises a first surface and a second surface, and at least one groove disposed in the first surface of the sheet wall, wherein the groove does not extend through the sheet wall to the second surface;

b. disposing a therapeutic agent in at least a portion of the groove;

c. disposing a coating over at least a portion of the first surface such that at least a portion of the therapeutic agent disposed in the groove is covered by the coating;

d. forming from the sheet, having the coating disposed on the first surface, a sidewall structure comprising a plurality of struts and gaps, wherein at least one of the struts comprises an top surface, a bottom surface, a first side surface and a cavity, wherein the cavity comprises a portion of the groove containing the therapeutic agent, and an opening that is in fluid communication with the first side surface, and wherein the opening is at least partially exposed.

Paragraph 3. The method of paragraph 2 further comprising forming the sidewall structure into a tubular shape before or after the sidewall structure is form.
Paragraph 4. The method of Paragraph 2 further comprising forming the sidewall structure into a tubular shape before or after the sidewall structure is form.
Paragraph 5. A method for making an implantable stent comprising:

a. providing a flat sheet of material having a sheet wall, in which the sheet wall comprises a first surface and a second surface, and at least one groove disposed in the first surface of the sheet wall, wherein the groove does not extend through the sheet wall to the second surface;

b. disposing a filler material in at least a portion of the groove;

c. disposing a coating over at least a portion of the first surface such that at least a portion of the filler material disposed in the groove is covered by the coating;

d. forming from the sheet, having the coating disposed on the first surface, a sidewall structure comprising a plurality of struts and gaps, wherein at least one of the struts comprises a top surface, a bottom surface, a first side surface and a cavity, wherein the cavity comprises a portion of the groove containing the filler material, and an opening that is in fluid communication with the first side surface, and wherein the opening is at least partially exposed.

e. removing at least a portion of the filler material from the cavity; and

f. disposing a therapeutic agent in the cavity.

Paragraph 6. The method of paragraph 5, wherein the disposing of the therapeutic agent in the cavity comprises:

(1) disposing the sidewall structure between a first flat surface and a second flat surface;

(2) exposing the sidewall structure to a therapeutic agent; and

(3) allowing the therapeutic agent to enter the cavity.

Paragraph 7. The method of paragraph 6, wherein the therapeutic agent is contained in a composition that is capable of becoming solid and the sidewall structure is exposed to the composition and the composition is allowed to enter the cavity.
Paragraph 8. The method of paragraph 6, wherein the therapeutic agent is allowed to enter the cavity by being pressure forced into the cavity.
Paragraph 9. An implantable self-expanding stent comprising:

a. a self-expanding stent sidewall structure comprising a plurality of struts and gaps in the sidewall structure, wherein at least one of the struts comprises an abluminal surface, a luminal surface, and a first side surface; and

b. a first coating, which comprises a polymer or a therapeutic agent, disposed on at least a portion of the first side surface, wherein the abluminal and luminal surfaces are substantially free of any coating.

Paragraph 10. The stent of paragraph 9, wherein the strut further comprises a second side surface opposite the first side surface and second coating disposed on at least a portion of the second side surface.
Paragraph 11. A method for making an implantable self-expanding stent comprising:

a. providing an implantable self-expanding stent having a stent sidewall structure comprising a plurality of struts and gaps in the sidewall structure, wherein at least one strut comprises an abluminal surface, a luminal surface, and a first side surface;

b. disposing the stent on a mandrel;

c. applying a coating composition comprising a therapeutic agent or a

polymer onto a flat surface;

d. rolling the stent disposed on the mandrel in the coating composition applied to the flat surface to apply the coating composition to the first side surface; and

e. ensuring that the abluminal surface and the luminal surface are substantially free of the coating composition.

Paragraph 12. The method of Paragraph 11, wherein the stent is rolled with sufficient pressure such that the coating composition is applied to the first side surface while the abluminal surface is made substantially free of the coating composition.
Paragraph 13. A method for making an implantable stent comprising:

a. providing a tube having a tubular wall having a longitudinal axis, in which the tubular wall comprises an abluminal surface and a luminal surface, and at least one groove disposed in the abluminal surface of the tubular wall, wherein the groove does not extend through the tubular wall to the luminal surface;

b. disposing a therapeutic agent in at least a portion of the groove;

c. disposing a coating over at least a portion of the abluminal surface such that at least a portion of the therapeutic agent disposed in the groove is covered by the coating;

d. forming from the tube, having the coating disposed on the abluminal surface, a stent having a stent sidewall structure comprising a plurality of struts and gaps, wherein at least one of the struts comprises an abluminal surface, a luminal surface, a first side surface and a cavity, wherein the cavity comprises a portion of the groove containing the therapeutic agent, and an opening that is in fluid communication with the first side surface, and wherein the opening is at least partially exposed.

The description contained herein is for purposes of illustration and not for purposes of limitation. The methods and devices described herein can comprise any feature described herein either alone or in combination with any other feature(s) described herein. Changes and modifications may be made to the embodiments of the description. Indeed, various modifications, in addition to those shown and described herein, will become apparent to those skilled in the art from the foregoing description and accompanying drawings using no more than routine experimentation. Such modifications and equivalents are intended to fall within the scope of the appended claims.

All publications, patents and patent applications mentioned in this specification are herein incorporated by reference into specification to the same extent as if each individual publication, patent or patent application was specifically and individually indicated to be incorporated herein by reference. Citation or discussion of reference herein shall not be construed as an admission that such is prior art.

What is claimed is:

1. An implantable stent comprising:
a. a stent sidewall structure comprising a plurality of struts and gaps in the sidewall structure, wherein at least one strut comprises an abluminal surface, a luminal surface, and a first side surface, and wherein the strut comprises a first material;
b. at least one cavity disposed in the at least one strut, wherein the cavity has a first opening that is in fluid communication with the abluminal surface and a second opening that is in fluid communication with the first side surface, wherein the cavity does not extend through the strut to the luminal surface;
c. a therapeutic agent disposed in the cavity; and
d. a coating disposed over at least a portion of the first opening of the cavity, wherein the second opening of the cavity is at least partially exposed.

2. The stent of claim 1, wherein at least a portion of the first opening is in contact with at least a portion of the second opening.

3. The stent of claim 1, wherein the strut further comprises a second side surface opposite the first side surface and the cavity has a third opening that is in fluid communication with the second side surface; wherein the third opening of the cavity is at least partially exposed.

4. The stent of claim 3, wherein the first or second side surface is a cut-surface.

5. The stent of claim 1, wherein the coating is disposed over the entire first opening of the cavity.

6. The stent of claim 1, wherein the coating comprises a material that prevents the release of the therapeutic agent from the cavity through the coating.

7. The stent of claim 1, wherein the coating comprises a material having a plurality of pores therein that allows for the release of the therapeutic agent from the cavity through the coating.

8. The stent of claim 3, wherein at least a portion of the first opening is in contact with at least a portion of the second opening and at least a portion of the third opening; wherein the therapeutic agent comprises an anti-restenosis agent, and wherein the coating prevents release of the anti-restenosis agent from the cavity through the coating.

9. An implantable stent comprising:
a. a stent sidewall structure comprising a plurality of struts and gaps in the sidewall structure, wherein at least one strut comprises an abluminal surface, a luminal surface, a first side surface, which is a cut surface, and a second side surface, which is a cut surface, opposite the first side surface;
b. at least one cavity disposed in the at least one strut, wherein the cavity has a first opening that is in fluid communication with the first side surface and a second opening that is in fluid communication with the second side surface, wherein the first and second opening of the cavity are at least partially exposed; and wherein the cavity does not have any opening that is in fluid communication with the abluminal surface or the luminal surface; and
c. a therapeutic agent disposed in the cavity.

10. The stent of claim 9, further comprising a coating, which comprises a polymer or a therapeutic agent, disposed on a portion of one or more of the abluminal surface, the luminal surface, the first side surface or the second side surface.

11. A method for making an implantable stent comprising:
a. providing a tube having a tubular wall having a longitudinal axis, in which the tubular wall comprises an abluminal surface and a luminal surface, and at least one groove disposed in the abluminal surface of the tubular wall, wherein the groove does not extend through the tubular wall to the luminal surface;
b. disposing a filler material in at least a portion of the groove;
c. disposing a coating over at least a portion of the abluminal surface such that at least a portion of the filler material disposed in the groove is covered by the coating;
d. forming from the tube, having the coating disposed on the abluminal surface, a stent having a stent sidewall structure comprising a plurality of struts and gaps, wherein at least one of the struts comprises an abluminal surface, a luminal surface, a first side surface and a cavity, wherein the cavity comprises a portion of the groove containing the filler material, and an opening that is in fluid communication with the first side surface, and wherein the opening is at least partially exposed.
e. removing at least a portion of the filler material from the cavity; and
f. disposing a therapeutic agent in the cavity.

12. The method of claim 11, wherein the disposing of the therapeutic agent in the cavity comprises:
(1) disposing the stent on an expandable mandrel;
(2) placing the stent disposed on the mandrel in a tube having an inner wall;
(3) expanding the mandrel so that at least a portion of the stent contacts the inner wall of the tube;
(4) exposing the stent to a therapeutic agent; and
(5) allowing the therapeutic agent to enter the cavity.

13. The method of claim 12, wherein the therapeutic agent is contained in a composition that is capable of becoming solid and the stent is exposed to the composition and the composition is allowed to enter the cavity.

14. The method of claim 12, wherein the composition is allowed to enter the cavity by being pressure forced into the cavity.

15. The method of claim 11, wherein the disposing of the therapeutic agent in the cavity comprises:
(1) disposing the stent on an rigid mandrel;
(2) placing the stent disposed on the mandrel in a tube having an inner
wall;
(3) closing the tube onto the stent so that at least a portion of the stent contacts the inner wall of the tube;
(4) exposing the stent to a therapeutic agent; and
(5) allowing the therapeutic agent to enter the cavity.
